(19) United States
(12) Patent Application Publication
(10) Pub. No.: US 2023/0248337 A1
(43) Pub. Date: Aug. 10, 2023

(54) SYSTEMS AND METHODS FOR GRATING LOBE REDUCTION IN ULTRASOUND IMAGING

(71) Applicant: KONINKLIJKE PHILIPS N.V., EINDHOVEN (NL)

(72) Inventors: Faik Can Meral, Mansfield, MA (US); Jean-Luc Francois-Marie Robert, Cambridge, MA (US); Francois Guy Gerard Marie Vignon, Andover, MA (US); Jun Seob Shin, Winchester, MA (US); Iason Zacharias Apostolakis, Cambridge, MA (US); Seyedali Sadeghi, Melrose, MA (US)

(21) Appl. No.: 18/010,001

(22) PCT Filed: Jun. 16, 2021
(86) PCT No.: PCT/EP2021/066191

§ 371 Date: Dec. 13, 2022

Related U.S. Application Data
(60) Provisional application No. 63/043,252, filed on Jun. 24, 2020.

(51) Int. Cl. A61B 8/08; G01S 15/89; G01S 7/52; G10K 11/34

(52) U.S. Cl. CPC A61B 8/5269; G01S 15/8915; G01S 7/52092; G10K 11/343

(57) ABSTRACT

In some examples, received signals from certain multilines may be selectively filtered to remove aliased frequencies that may result in grating lobes in ultrasound images. In some examples, a transmit beam may be shaped to reduce spatial frequencies in received signals. In some examples, the width of the transmit beam may be adjusted based on a frequency of the transmit signal. In some examples, a focal depth of the transmit beam may be adjusted based on a frequency of the transmit signal.

### TECHNICAL FIELD

This application relates to reducing grating lobe artifacts in ultrasound imaging. More specifically, this application relates to filtering of multilines and transmit beam shaping for reduction of grating lobe artifacts in ultrasound imaging.

### BACKGROUND

Grating lobes are artifacts in ultrasound imaging due to undersampling of spatial frequencies by transducer arrays, which results in aliasing of the undersampled frequencies. Minimum array spacing, (e.g., the distance between two elements), also known as the pitch, should be equal or less than λ/2, where λ is the wavelength of the ultrasound signal. In the images from arrays where this array spacing criteria is not met, grating lobes may be observed, for example, when the beam is steered beyond a certain angle.

Several methods have been proposed for grating lobe reduction in ultrasound images. Methods based on cross correlation of signals from adjacent transducer elements try to detect the phase shift of the signals larger than half the wavelength and correct for these shifts. However, these methods are computationally expensive and less effective when grating lobe signals and tissue signals exist in the same time. Methods based on phase-coherence of ultrasound signals across the aperture is effective in reducing the contribution of signals whose phases are not coherent, such as sidelobes and whose phases are not fully coherent across the wideband frequencies of ultrasound, such as grating lobes. However, these methods require a tuning parameter and can be too aggressive, which causes loss of the tissue signal. Accordingly, improved methods of grating lobe reduction is desired.

### SUMMARY

Techniques to reduce grating lobes by filtering only on the multilines and/or steering angles where the grating lobes are prominent are disclosed herein. Examples may take advantage of the Nyquist steering angle for a particular frequency and determine the spatial frequencies/multilines within the Nyquist steering angle limits. In some examples, at lower temporal frequencies more multilines can be used, but at higher frequencies the number of multilines that are be used is decreased which may reduce the production of grating lobes.

In some examples, a shape of a transmit beam may be adjusted by having a frequency-dependent aperture. For example, the beam may be narrow for high frequencies and wide for low frequencies to reduce grating lobes. In some examples, a focal depth of the transmit beam may be adjusted based on frequency.

According to examples of the present disclosure, an ultrasound imaging system may include a transducer array configured for transmitting ultrasound signals, receiving echoes responsive to the ultrasound signals, and providing receive signals corresponding to the echoes for a plurality of multilines, and a processor configured to determine a maximum steering angle for the transducer array, wherein the maximum steering angle is based, at least in part, on a pitch of the transducer array and a frequency of the ultrasound signals, determine a steering angle for individual ones of the plurality of multilines, wherein the steering angle is based, at least in part, on the pitch of the transducer array, and filter the receive signals corresponding to one or more of the plurality of multilines that have steering angles greater than the maximum steering angle before processing the receive signals into ultrasound image data.

According to examples of the present disclosure, a method may include transmitting an ultrasound signal with a transducer array, receiving echoes responsive to the ultrasound signal at the transducer array, generating receive signals for a plurality of multilines with the transducer array, determining a maximum steering angle based, at least in part, on a frequency of the ultrasound signal and a pitch of the transducer array, determining a steering angle for individual ones of the plurality of multilines, and filtering the receive signals corresponding to one or more of the plurality of multilines that have steering angles greater than the maximum steering angle before processing the receive signals into ultrasound image data.

According to examples of the present disclosure, an ultrasound imaging system may include a transducer array configured for transmitting a transmit beam comprising ultrasound signals, receiving echoes responsive to the ultrasound signals, and providing receive signals corresponding to the echoes for a plurality of multilines and a controller configured to provide control signals to the transducer array to cause the transducer array to transmit the ultrasound signals such that a width of the transmit beam is adjusted based on frequencies of the ultrasound signals, wherein the width of the transmit beam is wider for low frequencies and narrower for high frequencies.

### BRIEF DESCRIPTION OF THE DRAWINGS

FIG. 1 is a block diagram of an ultrasound imaging system arranged in accordance with examples of the present disclosure.

FIG. 2 is a block diagram illustrating an example processor in accordance with examples of the present disclosure.

FIG. 3 is an example plot of temporal frequency versus steering angle for a simulated 1D transducer array in accordance with principles of the present disclosure.

FIG. 4 shows multiple plots of frequency versus a number of multilines that are filtered by a filter according to examples of the present disclosure.

FIG. 5 shows images from a diverging wave simulation filtered according to examples of the present disclosure.

FIG. 6 shows images of a heart phantom filtered according to examples of the present disclosure.

FIGS. 7A and 7B illustrate transmit beams shaped according to examples of the present disclosure.

FIGS. 8A and 8B illustrate transmit waveforms for adjusting a width of a transmit beam according to examples of the present disclosure.

FIG. 9 shows an example plot of a multiline beam-fan.

### DESCRIPTION

The following description of certain exemplary examples is merely exemplary in nature and is in no way intended to limit the disclosure or its applications or uses. In the following detailed description of examples of the present systems and methods, reference is made to the accompanying drawings which form a part hereof, and in which are shown by way of illustration specific examples in which the described apparatuses, systems and methods may be practiced. These examples are described in sufficient detail to enable those skilled in the art to practice the presently disclosed apparatuses, systems and methods, and it is to be understood that other examples may be utilized and that structural and logical changes may be made without departing from the spirit and scope of the present disclosure. Moreover, for the purpose of clarity, detailed descriptions of certain features will not be discussed when they would be apparent to those with skill in the art so as not to obscure the description of the present disclosure. The following detailed description is therefore not to be taken in a limiting sense, and the scope of the present apparatuses, systems and methods is defined only by the appended claims.

In ultrasound imaging, elements of a transducer array are used to transmit one or more ultrasound signals into an object. The trajectories of the transmitted ultrasound signals may be referred to as ‘transmit lines.’ Some or all of the elements of the transducer array may be used for each transmit event. Echoes responsive to the transmitted ultrasound signals may be received by the transducer array from one or more points along one or more trajectories, which may be referred to as ‘receive lines.’ Some or all of the elements of the transducer array may be used for each reception event. Signals generated from the echoes may undergo beamforming and/or other processing to determine the receive line(s) the signals correspond to and construct an ultrasound image of the object. The trajectories of the transmit lines and the receive lines may be the same in some cases. In some applications, a single receive line may be generated for each transmit line and/or transmit event to form the ultrasound image.

In some applications, signals may be processed for multiple receive lines for each transmit line and/or transmit event to form the ultrasound image, which may be referred to as multiline beamforming. A potential advantage of multiline beamforming is higher frame rates for a given line density. By reconstructing multiple, simultaneous receive lines for each transmit line and/or transmit event, it may be possible to obtain frame rates equivalent to the multiple of receive lines generated. Another potential advantage may be improved image quality. By reconstructing the same receive line from multiple transmits and averaging them, a receive line with higher signal-to-noise ratio and/or more spatial frequencies may be obtained. When multiline beamforming is used, the receive lines may be referred to as ‘multilines.’

Some ultrasound probes include transducer arrays that are two dimensional (2D) arrays. That is, the transducer array includes multiple transducer elements in two dimensions (e.g., x-y). While the transducer elements may be arranged in a variety of shapes, the most common are square, rectangular, and circular arrangements. A 2D array may permit more complex beam steering and/or improved resolution compared to 1D arrays. However, as the number of transducer elements in the array increases, the number of wires connecting each transducer element in the probe to an ultrasound imaging system increases. As the number of wires increases, the cable connecting the probe to the ultrasound imaging system may become too large and unwieldly for practical use. To decrease the number of wires between the probe and the ultrasound imaging system, some transducer arrays are organized into groups of transducer elements, referred to as patches or subarrays that are included in the larger array. Rather than individual transducer elements, patches of transducer elements may be selectively activated for transmitting ultrasound signals and/or receiving echoes. Some ultrasound probes that include a transducer array grouped into patches may include microbeamformers to perform initial beamforming (e.g., delay-and-sum beamforming) on signals for the patches. For example, each microbeamformer may apply pre-defined focusing and steering delays to signals for a patch of five transducer elements. Thus, instead of five wires, only one wire is required to transmit the combined signal (e.g., semi-beamformed signal) from the group of five transducer elements.

Microbeamformers work well with focused transmit beams, since the microbeamformers can be pre-programmed to focus and steer signals from the patches to a main axis of a focused transmit beam. However, when diverging wave and/or plane wave transmits are used to insonify a region of interest, for example, for fast imaging sequences, multiple beams are formed to cover a larger angular span. The microbeamformers can be used to steer the signals from the patches to form these multiple beams, but the spacing between the patches may not be optimal for steering the beam far from the original pre-programmed angles. That is, although the individual transducer elements may meet the λ2 pitch requirement, because the transducer elements organized into patches cannot be individually controlled, the pitch may effectively be the spacing between patches, not the individual transducer elements. Thus, the pitch of the patches may not meet the λ2 requirement, which in turn may cause grating lobe artifacts in the resulting image.

According to examples of the present disclosure, filtering techniques may be used that filter only multilines and/or steering angles where grating lobes are prominent. As will be explained further herein, the λ2 pitch requirement is frequency dependent. If a narrowband signal model can be assumed, given an undersampled transducer array, a location of a resulting grating lobe may be predicted. Thus, a frequency of the resulting grating lobe signals may be predicted if the main lobe and grating lobe locations are known. Therefore, for a given steering angle, a frequency band the grating lobe signals will leak into may be predicted. This frequency band may then be filtered out. While examples of the present disclosure are discussed with reference to ultrasound probes that utilize microbeamformers, the techniques disclosed herein may be applied to any transducer array that suffers from undersampling. Furthermore, although some of the examples disclosed herein refer to divergent and plane waves, techniques disclosed herein are not limited to a particular transmit wave regime.

According to examples of the present disclosure, aliasing due to undersampling may be reduced or avoided by altering a shape of a transmitted ultrasound beam to reduce or eliminate grating lobes. In some examples, transmit beam shaping techniques may be used that vary the transmit aperture based on the frequency of the transmit waveform. In some examples, transmit beam shaping techniques may be used that vary a frequency of the transmit waveform based on a focal depth of the transmit waveform.

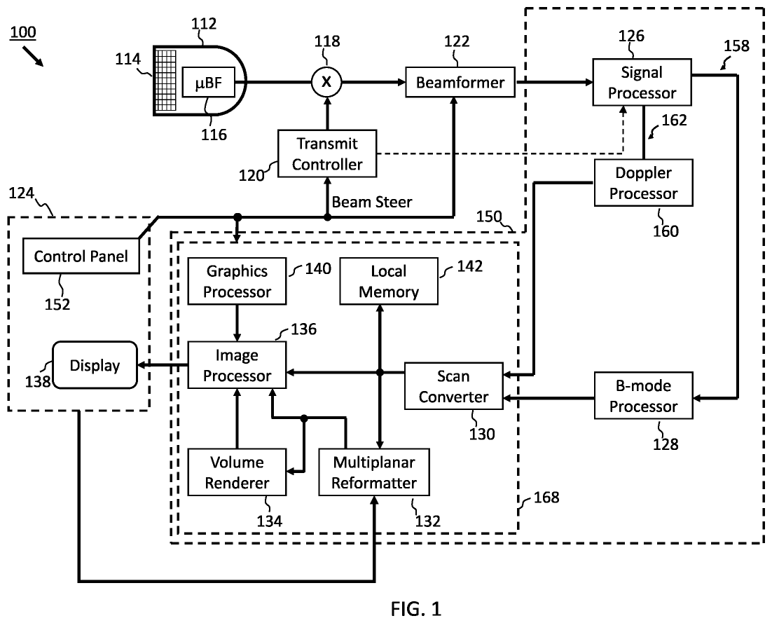

FIG. 1 shows a block diagram of an ultrasound imaging system 100 constructed in accordance with examples of the present disclosure. An ultrasound imaging system 100 according to the present disclosure may include a transducer array 114, which may be included in an ultrasound probe 112, for example an external probe or an internal probe such as an intravascular ultrasound (IVUS) catheter probe. In other examples, the transducer array 114 may be in the form of a flexible array configured to be conformally applied to a surface of subject to be imaged (e.g., patient). The transducer array 114 is configured to transmit ultrasound signals (e.g., beams, waves) and receive echoes (e.g., received ultrasound signals) responsive to the transmitted ultrasound signals. A variety of transducer arrays may be used, e.g., linear arrays, curved arrays, or phased arrays. The transducer array 114, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some examples, the transducer array 114 may be coupled to a microbeamformer 116, which may be located in the ultrasound probe 112, and which may control the transmission and reception of signals by the transducer elements in the array 114. In some examples, the microbeamformer 116 may control the transmission and reception of signals by active elements in the array 114 (e.g., an active subset of elements of the array that define the active aperture at any given time).

In some examples, the microbeamformer 116 may be coupled, e.g., by a probe cable or wirelessly, to a transmit/receive (T/R) switch 118, which switches between transmission and reception and protects the main beamformer 122 from high energy transmit signals. In some examples, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface.

In some examples, the transmission of ultrasonic signals from the transducer array 114 under control of the microbeamformer 116 is directed by the transmit controller 120, which may be coupled to the T/R switch 118 and a main beamformer 122. The transmit controller 120 may control the direction in which beams are steered (e.g., by providing control signals to the microbeamformer 116, transducer array 114, and/or individual elements of the transducer array 114). Beams may be steered straight ahead from (orthogonal to) the transducer array 114, or at different angles for a wider field of view.

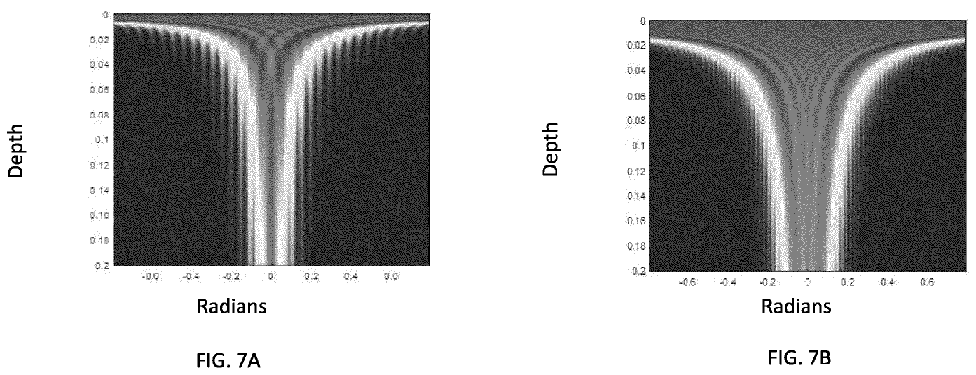

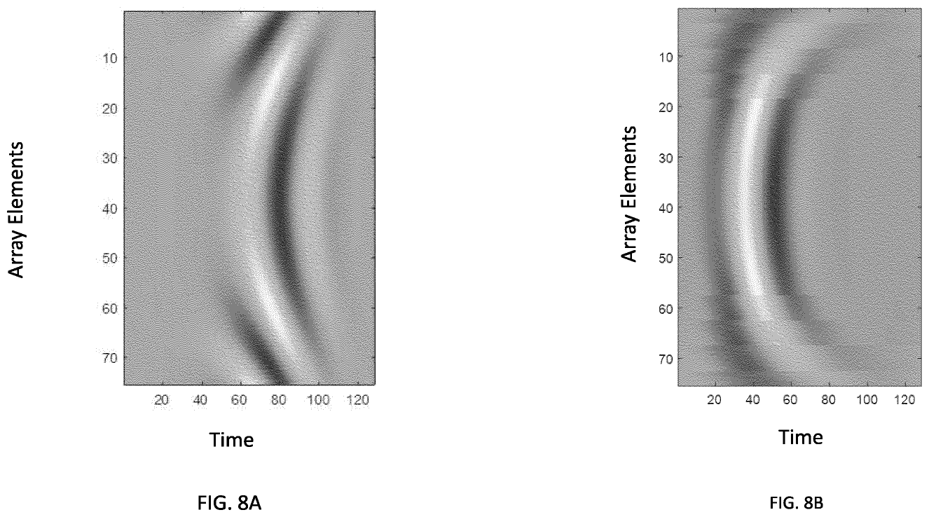

According to examples of the present disclosure, the transmit controller 120 may control a shape of the transmitted beams to reduce or eliminate grating lobe artifacts. As will be described in more detail with reference to FIGS. 7 and 8, in some examples the transmit controller 120 may adjust an aperture of the transmit beam based on the frequency or frequencies of the transmitted ultrasound signals of the transmit beam. For example, wider beams may be used for low frequencies of transmit beams and narrower beams may be used for high frequencies of transmit beams. In other examples, a focal depth of the transmit beam may be adjusted based on a frequency of the transmitted ultrasound signals of the transmit beam. For example, shallower focal depths may be used for lower frequencies and deeper focal depths may be used for higher frequencies.

In some examples, the transmit controller 120 may also be coupled to a user interface 124 and receive input from the user's operation of a user input device (e.g., user control). The user interface 124 may include one or more input devices such as a control panel 152, which may include one or more mechanical controls (e.g., buttons, sliders, etc.), touch sensitive controls (e.g., a trackpad, a touchscreen, or the like), and/or other known input devices.

In some examples, the partially beamformed signals produced by the microbeamformer 116 may be coupled to a main beamformer 122 where partially beamformed signals from individual patches of transducer elements may be combined into a fully beamformed signal. In some examples, microbeamformer 116 is omitted. In these examples, the transducer array 114 is under the control of the main beamformer 122, and the main beamformer 122 performs all beamforming of signals. In examples with and without the microbeamformer 116, the beamformed signals of the main beamformer 122 are coupled to processing circuitry 150, which may include one or more processors (e.g., a signal processor 126, a B-mode processor 128, a Doppler processor 160, and one or more image generation and processing components 168) configured to produce an ultrasound image from the beamformed signals (i.e., beamformed RF data).

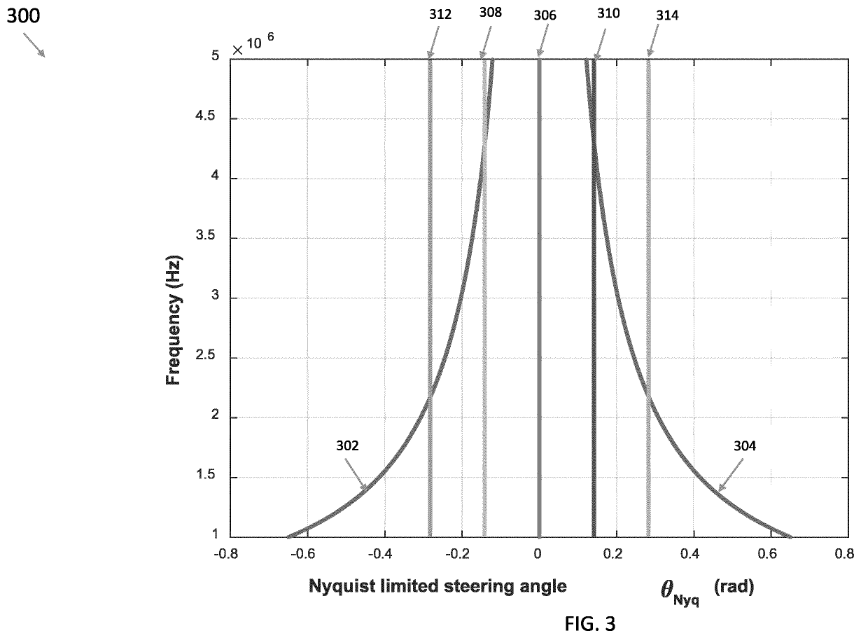

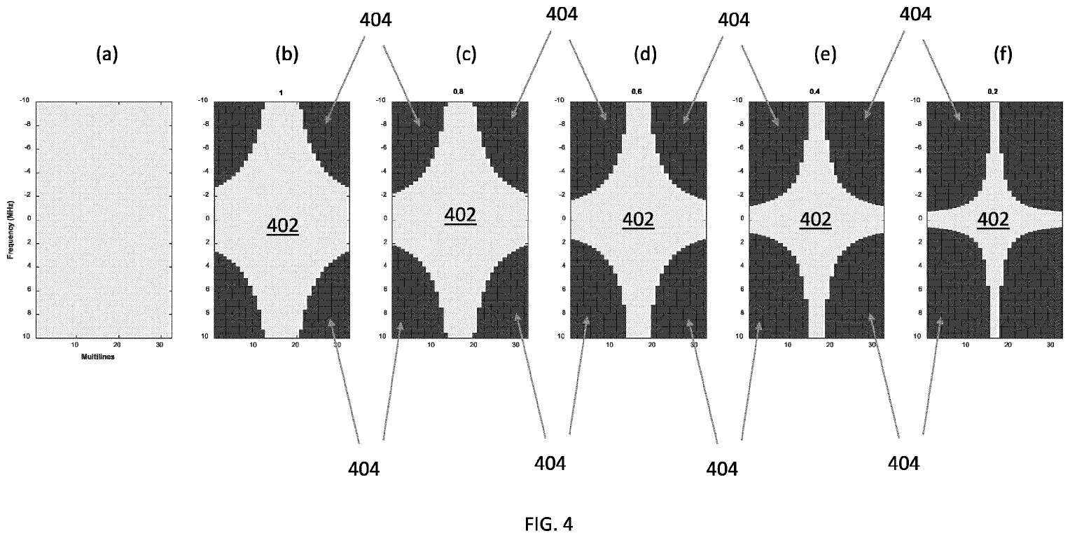

According to examples of the present disclosure, the signal processor 126 may be configured to filter signals corresponding grating lobe artifacts to reduce or eliminate the grating lobe artifacts due to undersampling/aliasing of the received ultrasound signals (e.g., echoes). In some examples, the signal processor 126 may selectively filter signals from one or more multilines and/or from particular steering angles. As described, with reference to FIGS. 3 and 4, the aliased frequencies may be associated with particular multilines and/or steering angles. Because the steering angle is controlled by the transmit controller 120, the steering angle may be known. Based on the steering angle of the transmit beam, the signal processor 126 may determine the location of the grating lobe artifacts. The signal processor 126 may use the location of the grating lobe artifacts to select the multilines to be filtered or otherwise removed from the beamformed signals.

Although not shown in FIG. 1, in some examples, an additional filter, which may be implemented in any suitable processor, may be included prior to the main beamformer 122. In these examples, this additional filter may selectively filter one or more channels of signals provided by the microbeamformer 116 before the channels are combined into multilines to reduce or substantially remove grating lobe artifacts that may be associated with those channels. The underlying principles for filtering grating lobes, whether at the signal processor 126 stage or earlier in the signal path, such as by an additional filter preceding the main beamformer, remain the same as described throughout this disclosure.

The signal processor 126 may also be configured to process the received beamformed RF data in various ways, such as bandpass filtering, decimation, I and Q component separation, and harmonic signal separation. The signal processor 126 may also perform additional signal enhancement such as speckle reduction, signal compounding, and electronic noise elimination. The processed signals (also referred to as I and Q components or IQ signals) may be coupled to additional downstream signal processing circuits for image generation. The IQ signals may be coupled to a plurality of signal paths within the system, each of which may be associated with a specific arrangement of signal processing components suitable for generating different types of image data (e.g., B-mode image data, Doppler image data). For example, the system may include a B-mode signal path 158 which couples the signals from the signal processor 126 to a B-mode processor 128 for producing B-mode image data.

The B-mode processor 128 can employ amplitude detection for the imaging of structures in the body. The B-mode processor 128 may generate signals for tissue images and/or contrast images. The signals produced by the B-mode processor 128 may be coupled to a scan converter 130 and/or a multiplanar reformatter 132. The scan converter 130 may be configured to arrange the echo signals from the spatial relationship in which they were received to a desired image format. For instance, the scan converter 130 may arrange the echo signal into a two-dimensional (2D) sector-shaped format, or a pyramidal or otherwise shaped three-dimensional (3D) format.

In some examples, the system may include a Doppler signal path 162 which couples the output from the signal processor 126 to a Doppler processor 160. The Doppler processor 160 may be configured to estimate the Doppler shift and generate Doppler image data. The Doppler image data may include color data which is then overlaid with B-mode (i.e. grayscale) image data for display. The Doppler processor 160 may be configured to filter out unwanted signals (i.e., noise or clutter associated with non-moving tissue), for example using a wall filter. The Doppler processor 160 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function (e.g., R1) and Doppler power estimation is based on the magnitude of the lag-zero autocorrelation function (e.g., R0). The velocity estimations may be referred to as color Doppler data and the power estimations may be referred to as power Doppler data. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some examples, the velocity and power estimates (e.g., the color and power Doppler data) may undergo further threshold detection to further reduce noise, as well as segmentation and post-processing such as filling and smoothing. The velocity and/or power estimates may then be mapped to a desired range of display colors and/or intensities in accordance with one or more color and/or intensity maps. The map data, also referred to as Doppler image data, may then be coupled to the scan converter 130, where the Doppler image data may be converted to the desired image format to form a color Doppler or a power Doppler image.

The multiplanar reformatter 132 can convert echoes which are received from points in a common plane (e.g., slice) in a volumetric region of the body into an ultrasonic image (e.g., a B-mode image) of that plane, for example as described in U.S. Pat. No. 6,443,896 (Detmer). In some examples, the user interface 124 may be coupled to the multiplanar reformatter 132 for selection and control of a display of multiple multiplanar reformatted (MPR) images. In some examples, the plane data of the multiplanar reformatter 132 may be provided to a volume renderer 134. The volume renderer 134 may generate (also referred to as render) an image (also referred to as a projection, rendering, or 3D scene) of the 3D dataset as viewed from a given reference point, for example as described in U.S. Pat. No. 6,530,885 (Entrekin et al.).

Output from the scan converter 130 (e.g., B-mode images, Doppler images), the multiplanar reformatter 132, and/or the volume renderer 134 (e.g., volumes, 3D scenes) may be coupled to an image processor 136 for further enhancement, buffering and temporary storage before being displayed on an image display 138. In some examples, a Doppler image may be overlaid on a B-mode image of the tissue structure by the scan converter 130 and/or image processor 136 for display.

A graphics processor 140 may generate graphic overlays for display with the images. These graphic overlays may contain, for example, standard identifying information such as patient name, date and time of the image, imaging parameters, and the like. For these purposes the graphics processor 140 may be configured to receive input from the user interface 124, such as a typed patient name or other annotations

The system 100 may include local memory 142. Local memory 142 may be implemented as any suitable non-transitory computer readable medium (e.g., flash drive, disk drive). Local memory 142 may store data generated by the system 100 including images, 3D models, executable instructions, inputs provided by a user via the user interface 124, or any other information necessary for the operation of the system 100.

As mentioned previously system 100 includes user interface 124. User interface 124 may include display 138 and control panel 152. The display 138 may include a display device implemented using a variety of known display technologies, such as LCD, LED, OLED, or plasma display technology. In some examples, display 138 may comprise multiple displays. The control panel 152 may be configured to receive user inputs (e.g., steering angle, filter aggressiveness, etc.). The control panel 152 may include one or more hard controls (e.g., buttons, knobs, dials, encoders, mouse, trackball or others). In some examples, the control panel 152 may additionally or alternatively include soft controls (e.g., GUI control elements or simply, GUI controls) provided on a touch sensitive display. In some examples, display 138 may be a touch sensitive display that includes one or more soft controls of the control panel 152.

In some examples, various components shown in FIG. 1 may be combined. For instance, image processor 136 and graphics processor 140 may be implemented as a single processor. In another example, the Doppler processor 160 and B-mode processor 128 may be implemented as a single processor. In some examples, various components shown in FIG. 1 may be implemented as separate components. For example, signal processor 126 may be implemented as separate signal processors for each imaging mode (e.g., B-mode, Doppler). In some examples, one or more of the various processors shown in FIG. 3 may be implemented by general purpose processors and/or microprocessors configured to perform the specified tasks. In some examples, one or more of the various processors may be implemented as application specific integrated circuits. In some examples, one or more of the various processors (e.g., image processor 136) may be implemented with one or more graphical processing units (GPU).

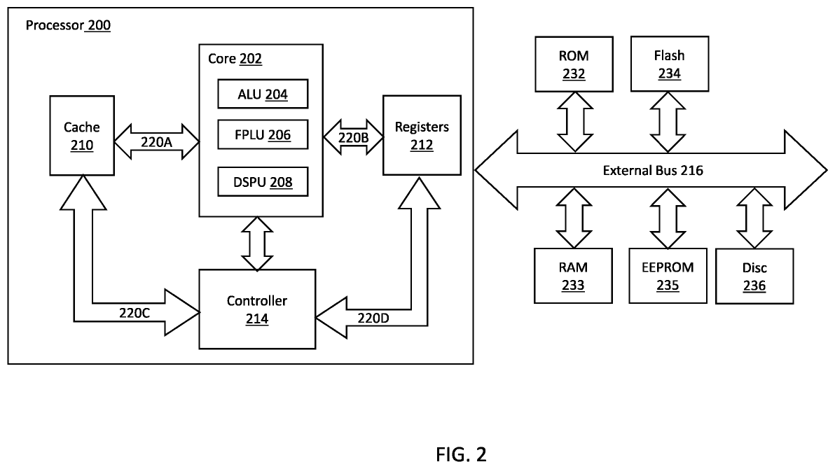

FIG. 2 is a block diagram illustrating an example processor 200 in accordance with examples of the present disclosure. Processor 200 may be used to implement one or more of the processors described herein, for example, image processor 136 and/or signal processor 126 shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific integrated circuit (ASIC) where the ASIC has been designed to form a processor, or a combination thereof.

The processor 200 may include one or more cores 202. The core 202 may include one or more arithmetic logic units (ALU) 204. In some examples, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some examples the registers 212 may be implemented using static memory. The register may provide data, instructions and addresses to the core 202.

In some examples, processor 200 may include one or more levels of cache memory 210 communicatively coupled to the core 202. The cache memory 210 may provide computer-readable instructions to the core 202 for execution. The cache memory 210 may provide data for processing by the core 202. In some examples, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., control panel 152 and scan converter 130 shown in FIG. 1) and/or outputs from the processor 200 to other processors and/or components included in the system (e.g., display 138 and volume renderer 134 shown in FIG. 1). Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache memory 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache memory 210, and/or register 212. The bus 216 may be coupled to one or more components of the system, such as display 138 and control panel 152 mentioned previously.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 233. RAM 233 may be a static RAM, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some examples, the external memories may be included in a system, such as ultrasound imaging system 100 shown in FIG. 1, for example local memory 142.

According to examples of the present disclosure, relationships between the temporal frequency domain and the spatial frequency domain as well as principles of beam steering may be used to reduce grating lobe artifacts by selectively filtering signals from received echoes responsive to a transmit beam. For beam steering, a time delay Δt between two consecutive transducer elements (e.g., two adjacent transducer elements in transducer array 114) may be given as:

$$Δ t = \frac{d}{c} sin ( θ ) \qquad Equation 1$$

Where θ is the steering angle, d is the element spacing, and c is the speed of sound in tissue. For a narrowband wave with temporal angular center frequency of ω, this expression may be provided in phase terms:

Δϕ=kdsin(θ) Equations 2

Where Δϕ=ωΔt and k is the wave-number (spatial frequency, k=2π/λ, λ is the associated spatial wavelength) which is analogous to the temporal angular frequency ω. The Nyquist-Shannon sampling theorem provides that the sampling frequency should be at least twice the highest frequency in the signal to avoid aliasing. For spatial coordinates and wave-number k this means the maximum value Δϕcan have is π for proper sampling of the signal. Thus, the Nyquist limit for the steering angle θ_{Nyq} of a beam may be provided as:

θ_{Nyq}=sin⁻¹(λ/2d) Equation 3

In other words, for transducer elements having a pitch of λ/2, beams steered beyond θ_{Nyq} (e.g., beams with steering angles greater than the Nyquist limit) may cause signals to be undersampled, causing aliasing and the resulting grating lobe artifacts. Although Equations 1-3 were described in reference to individual transducer elements, when microbeamformers are used, the pitch of patches of transducer elements may be used. Substituting the spatial wavelength term λ with its corresponding temporal frequency ω and 1540 m/s for the speed of sound in tissue c, θ_{Nyq} may be plotted for a frequency range of interest. The frequency range of interest may be a range of transmit frequencies the transducer array is capable of producing or a range of transmit frequencies used for a particular type of imaging.

FIG. 3 is an example plot of temporal frequency versus steering angle for a simulated 1D transducer array in accordance with principles of the present disclosure. In this specific example the 1D transducer array includes 16 5-element microbeamformers across the lateral dimension. The multiline settings are based on a 10 transmit diverging wave sequence to cover a sector of ˜90° and the resulting receive line spacing of the multilines is 0.0176742 rad. In plot 300, the curves 302, 304 are plots of θ_{Nyq}, within a 1-5 MHz frequency range. The vertical line 306 indicates a zero degree steering of the beam. In some applications, vertical line 306 may correspond to a multiline directly in front of a transducer element (or patch) of the 1D array that transmitted an ultrasound signal directly in front of the transducer element (or patch) in a direction perpendicular to the face of the 1D array (e.g., zero degree steering). Vertical lines 308, 310 indicate where sixteen multilines extend. These additional multilines may correspond to multilines offset from the transducer element (or patch) that transmitted the ultrasound signal and/or at an angle relative to the transmitted beam (e.g., the steering angle). Similarly vertical lines 312, 314 indicate the extent of thirty-two multilines.

As shown by the location of intersection of the vertical lines 312, 314 with curves 302, 304, respectively, for frequencies above 2.1 MHz, 32 multiline cases start to fall beyond the Nyquist limit and into the aliasing regime (e.g., the farthest multiline from the center may be located at approximately 0.28 radians, as indicated by vertical lines 312 and 314). At these frequencies the array becomes undersampled and further steering the beam will degrade the image quality. Similarly, 4.3 MHz is the Nyquist limit for the farthest lines of the 16 multiline case, where the farthest multiline from the center may be at approximately 0.14 radians, as indicated by vertical lines 308 and 310.

In embodiments according to the present disclosure, a low pass filter may be used for beams that are steered beyond target steering angles, such as Nyquist limit steering angles. The high frequency bands of received signals from these beams contain aliased information from different spatial locations. As described herein, the relationship between temporal and spatial frequencies may be used to generate a filter that is angle dependent and/or multiline dependent. In other words, the lower temporal frequencies where grating lobes are far away (e.g., 2 MHz), an ultrasound system including the simulated probe of FIG. 3 may use up to 32 multilines to fully take advantage of various methods of multiline beamforming, such as retrospective transmit beam compounding (XBR) to generate an ultrasound image along transmit lines, while for higher temporal frequencies (e.g., 4 MHz), only 16 multilines may be used to avoid grating lobes. The number of multilines may be gradually changed between the maximum and minimum number of multilines (e.g., 32 and 1, respectively or 32 and 16, respectively). In some applications, this may provide an acceptable trade-off between XBR gain and grating lobe rejection at each frequency. In some examples, frequency dependent XBR weights at the quadrature bandpass filter (QBP) stage of signal processing (e.g., by signal processor 126) could reject the multilines, or higher frequency bands of the multilines, that are not to be used.

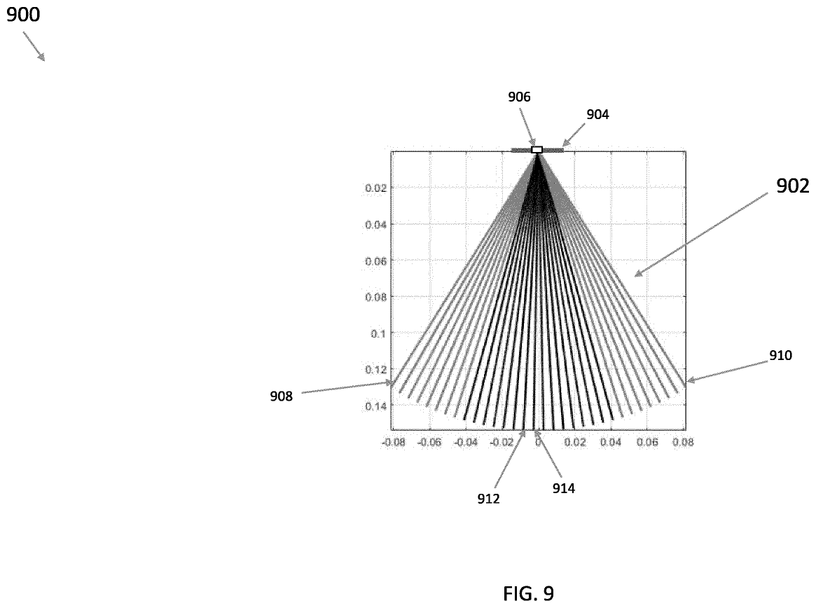

FIG. 4 shows multiple plots of frequency versus a number of multilines that are filtered by a filter according to examples of the present disclosure. In FIG. 4, the y-axis is the frequency axis (MHz) and x-axis is the multilines (number of multilines) for an example 1D transducer array. For each transducer element, the multilines may form a “fan” originating at the transducer element. An example plot 900 of a multiline beam-fan 902 is shown in FIG. 9. The multiline beam-fan 902 is emitted from a transducer element 906 of a transducer array 904. Transducer array 904 may be included in transducer array 112 in some examples. As shown in FIG. 9, Multiline 0 908 may be a line received at a far edge of a multiline beam-fan and multiline 31 910 may be a line received at an opposite edge of the multiline beam-fan 902 while multilines 15-16 912, 914 may be near a center of the multiline beam-fan 902. Thus, multilines 15-16 912, 914 may have a steering angle close or equal to zero in reference to the transducer element 906 whereas multilines 0 and 31 908, 910 may have the largest steering angles with reference to the transducer element 906. The number of multilines in this example are provided for illustration only and in other examples a different number of multilines may be used.

Returning to FIG. 4, plot (a) illustrates the case where no anti-alias (e.g., low pass) filter is applied and all the frequencies for all multilines are allowed to pass through a signal processor (e.g., signal processor 126). However, for beams whose nominal frequency at a center frequency of the transducer array, the multilines at the edges of the multiline beam-fan may have an angle above the Nyquist limit θ_{Nyq}. Thus, higher frequencies in the array domain (e.g., spatial domain) may be undersampled, leading to grating lobes. In plots (b)-(f), a low-pass filter is applied to filter signals from some of the multilines which may reduce grating lobes. The pass bands 402 (light region) and stop bands 404 (dark regions) may be specific to each multiline. In some examples, the pass bands 402 are based on the steering angle of the multiline, which may be based, at least in part, on the spacing between transducer elements and/or patches of transducer elements, and the frequency of the ultrasound signal as discussed with references to Equations 1-3. That is, for a given frequency, multilines having a steering angle above the Nyquist limit may be filtered. In some examples, the filtering may be specific to multilines based on the steering angle and may not interfere with any further processing of the multilines.

Although the Nyquist sampling frequency is the theoretical minimum sampling frequency necessary, in practice sampling frequencies higher than twice to Nyquist frequency are used. In the case of grating lobe filtering, this translates to more aggressive filters with lower cut-off frequencies, reducing the pass band 402. In some examples, instead of defining the new lower cut-off frequencies, a target steering angle θ_{Max} may be defined, which may be a fraction of θ_{Nyq}. The adjusted filter cut-offs (e.g., stop bands 404) are calculated based on a fraction r such that θ_{Max}=rθ_{Nyq}. The numbers above each plot (b)-(f) are different r values. The r value indicates what fraction of the ideal passband 402 (e.g., as calculated based at least in part on Equation 3) to use. Thus, r may have any value between and including zero and one. For example, in plot (b), r=1, so the entire ideal passband 402 may be used. The ideal passband 402 may indicate the multilines that include non-aliased frequencies up to the theoretical Nyquist limit. However, as noted, in practice, it may be desirable to stay below the Nyquist limit frequency and filter additional multilines to help ensure aliasing is avoided. Plots (c)-(f) show decreasing r values and a corresponding smaller fraction of the passband 402 is used. The filter becomes more aggressive as r decreases and an increasing number of multilines are filtered. In some examples, the r value may be preset in an ultrasound system. In other examples, a user may indicate the r value by providing a user input via a user interface (e.g., user interface 124).

In multiline beamforming, several multilines may be compounded, for example, using the XBR framework. A weight may be assigned to each multiline which determines a particular multiline's influence on a result of the compounding. The weights assigned to each multiline for compounding may be frequency dependent and/or steering angle dependent. In some examples, filtering by a signal processor (e.g., signal processor 126) according to examples of the present disclosure may include assigning weights to the multilines prior to compounding. Other processing steps performed by the signal processor may include filtering the multilines through one or multiple QBP filters, envelope and log-detection, and/or frequency compounding. In some examples these processing steps may be performed following the grating lobe filtering.

Although FIGS. 3-4 refer to a simulated 1D transducer array for explanatory purposes, the principles of the present disclosure are not limited to 1D arrays and may be applied to 2D transducer arrays. For 2D transducer arrays, the pitch of the array in both the x and y dimensions may need to be considered for the element spacing d in Equation 1 if they are not equal. A pitch between diagonally-spaced transducer elements and/or patches may also need to be considered in some applications. Similarly, a steering angle θ_{Nyq} may need to be calculated in two dimensions, typically with polar coordinates.

Returning to FIG. 1, while still referring to FIGS. 3 and 4, signal processor 126 may receive multiline signals from main beamformer 122. The signal processor 126 may be provided a pitch of the transducer array 114. The pitch may be provided via a user input through user interface 124 or through an identifier signal provided by the ultrasound probe 112. The signal processor 126 may be provided the frequencies of ultrasound signals transmitted by the transducer array 114 by the control panel 152 and/or transmit controller 120. Based, at least in part, on the pitch and transmit frequencies, the signal processor 126 may determine a maximum steering angle (e.g., Nyquist limit) of the transmit beam above which aliasing/undersampling will occur. The signal processor 126 may further be provided the steering angle of the ultrasound signals transmitted by the transducer array 114 by the control panel 152 and/or transmit controller 120. The signal processor 126 may determine which multiline(s) (if any) fall above the maximum steering angle based on the steering angle and a given frequency of the transmitted ultrasound signals. As noted herein, for example, with reference to FIG. 3, the steering angle associated with a multiline may be based, at least in part, on a pitch of the transducer elements and/or a pitch of patches of transducer elements (e.g., when microbeamformer 116 is included).

For multilines determined to be above the maximum steering angle for the given frequency, the signal processor 126 may filter the signals of those multilines. In some examples, filtering the signals from the multilines above the maximum steering angle may include reducing a power of the signals from the multilines for the given frequency, removing the signals from the multilines for the given frequency from further processing and/or not passing the signals to the Doppler processor 160 and/or B-mode processor 128. In some examples, filtering the signals from the multilines above the maximum steering angle for the given frequency may include applying a weight (e.g., 0, 0.1, 0.2) to the signal that reduces the signal's impact on compounding of the multiline signals or other further processing of the multiline signals.

In some examples, the signal processor 126 may also receive an r value. In some examples, the r value may be provided by a user input received from the user interface 124. In these examples, the signal processor 126 may filter multilines that are at a steering angle greater than rθ_{Nyq}.

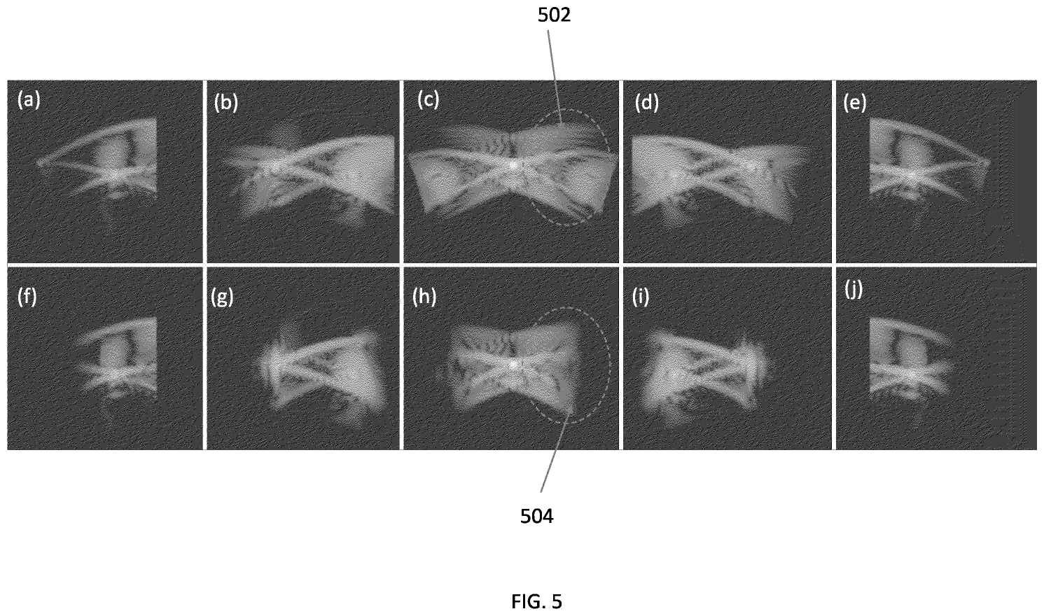

FIG. 5 shows images from a diverging wave simulation filtered according to examples of the present disclosure. Images (a) through (j) were generated by simulating three point targets and five diverging wave transmits. A focus of the diverging waves was set to negative 50 mm. The images from the individual diverging waves are shown side by side. Images (a) through (e) in the top row are the unfiltered images from each diverging wave transmit and images (f) through (j) in the bottom row are the corresponding filtered images filtered according to examples of the present disclosure. Filtering multilines reduces or eliminates the grating lobe artifacts of the points away from the transmit axis. All of the images in the bottom row show less grating lobes compared to their top row counterparts. For example, region 502 of image (c) contains more grating lobes than corresponding region 504 in image (h).

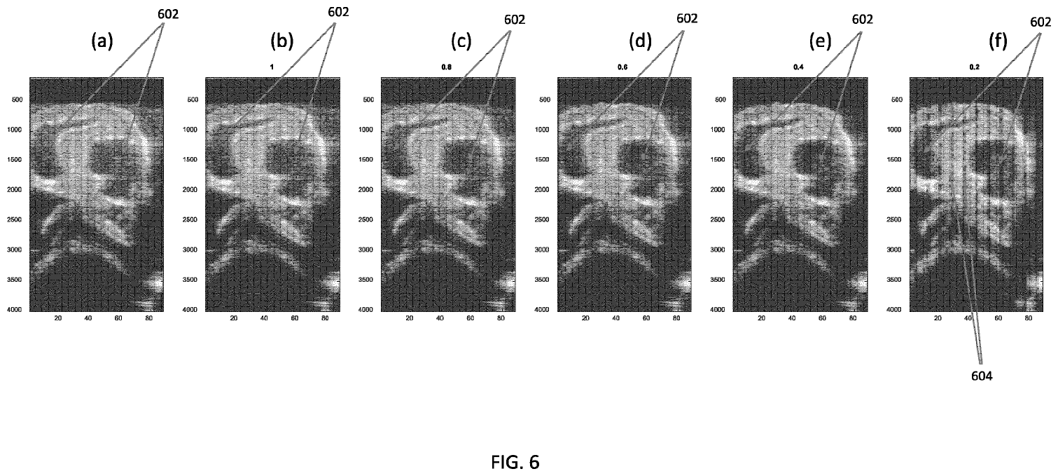

FIG. 6 shows images of a heart phantom filtered according to examples of the present disclosure. The images (a)-(f) are pre-scan converted images. All data for images (a)-(f) underwent QBP filtering, envelope and log detection, and frequency compounding. Data for images (b)-(f) further underwent grating lobe filtering according to examples of the present disclosure. The values above images (b)-(f) indicate the r value of the filter used to reduce or remove the grating lobe artifacts, where image (b) underwent the least aggressive filtering and image (f) underwent the most aggressive filtering.

The images (c)-(f) with r values lower than 1 show improved results in terms of grating lobe clutter reduction in the heart chambers 602 compared to images (a) and (b). However too aggressive values such as r=0.4 or r=0.2, as were used in images (e) and (f), may create jailbar artifacts 604. In these cases, filtering using these more aggressive filters may eliminate from the majority of the multilines some or most of the signals from a frequency band of interest.

In some applications, to reduce the jailbar effect, XBR processing following the grating lobe filtering operation may compound the aggressively filtered steered multilines (e.g. multilines 1-4 or 29-32) with one or more central multilines that are not filtered (e.g. multilines 15-18). This technique was used with images (e) and (f) to reduce the jailbar artifacts. However in the most aggressive setting of r=0.2 in image (f) there are still some lines left where the excessive filtering artefact is still present. In some examples, another technique to mitigate the jailbar effect may include renormalizing the multilines based on their pre and post filter powers prior to compounding. This means weighing the steered and filtered lower frequency multilines more to compensate for the power of missing signals at high frequencies. For example, if one half of the power of the signal is removed by filtering, the root mean square of the power may be added back to the signal to re-normalize.

While filtering received signals to reduce grating lobes as described above may be relatively straightforward to implement, even on existing ultrasound imaging system, filtering received signals is not ideal. Filtering out multilines that include aliased frequencies, is mitigating aliasing that has already occurred. A potentially better solution would be to prevent aliasing from occurring in the first place, for example, by reducing spatial frequencies that are prone to aliasing. In some examples, spatial frequencies present may be controlled, at least in part, by shaping a transmit beam transmitted by a transducer array (e.g., transducer array 114). Received signals originate from within the transmit beam, so changing a shape (e.g., width) of the transmit beam changes the spatial frequency content of the received signal. A narrower transmit beam leads to lower received spatial frequencies than a wider transmit beam.

The ideal transmit beam width is frequency dependent and follows Equations 1-3 as discussed with reference to FIG. 3. In some applications, a narrower transmit beam for higher temporal frequencies and a wider transmit beam width for lower frequencies may be desired. In some examples, this may be achieved by using frequency dependent transmit apertures. Implementing frequency dependent apertures may include transmitting different waveforms on different transducer elements of a transducer array. The waveform bandwidth would change from one element to another across the aperture. In some examples, a frequency dependent focal depth for the transmit beam may be used to adjust a width of the transmit beam. However, this technique may use more complicated waveforms than the previously discussed transmit beam shaping technique. In some examples, the transmit beam may be shaped based, at least in part, on control signals provided to the transducer array (e.g., transducer array 114) by a transmit controller (e.g., transmit controller 120).

FIGS. 7A and 7B illustrate transmit beams shaped according to examples of the present disclosure. In some examples, a width of the transmit beam may be adjusted based on temporal frequencies of the transmitted ultrasound signals to reduce aliasing and the subsequent grating lobe artifacts. FIG. 7A is an example transmit beam 702 for high frequencies of an ultrasound signal. FIG. 7B is an example transmit beam 704 for low frequencies of the ultrasound signal. Both FIGS. 7A and 7B are plotted in polar coordinates (e.g., depth versus radians). As shown, the width of transmit beam 702 is narrower than the transmit beam 704. The narrower transmit beam 702 may reduce the spatial frequencies resulting from high temporal frequency ultrasound signals, which may be aliased at the transducer array (e.g., when the pitch of the individual elements or patches is above the Nyquist limit). However, the wider transmit beam 704 may be used for lower temporal frequencies to permit a larger area and/or volume to be insonified during a transmit event.

Although reference is made to separate transmit beams 702 and 704, a transmit event may include “multiple” transmit beams in some examples. That is, transmit beams 702 and 704 may be transmitted by a transducer array at (or nearly at) the same time. Transducer elements (or patches of transducer elements) may transmit waveforms of ultrasound signals comprised of multiple frequencies and/or different transducer elements (or patches) may transmit waveforms of ultrasound signals with different frequencies than other transducer elements (or patches). For example, for a transmit event, some patches may transmit only lower frequencies, some patches may only transmit higher frequencies, while some patches may transmit a range of frequencies such that the transmit event includes transmit beams shaped for each of the frequencies and/or frequency ranges. In some examples, the transmit beams shaped for different frequencies may at least partially overlap spatially and/or temporally.

FIGS. 8A and 8B illustrate transmit waveforms for adjusting a width of a transmit beam according to examples of the present disclosure. In some examples, a focal depth of the transmit beam may be adjusted based on temporal frequencies of the transmitted ultrasound signals to reduce aliasing and the subsequent grating lobe artifacts. FIG. 8A is an example transmit waveform 802 emitted from a 1D transducer array. The X-axis is time (time samples) and the Y-axis is the transducer elements of the array. The transmit waveform 802 was generated by varying a focal depth from −70 mm for low temporal frequencies and −140 mm for the highest temporal frequencies of the transmitted ultrasound signal. This translates into adjusting the phase of the emitted wavefront to vary with frequency.

In some examples, instead of adjusting the phase of the ultrasound signals to alter the focal depth, and thus the width of the transmit beam, an aperture of the transducer array may be adjusted to alter the width of the transmit beam. FIG. 8B is an example transmit waveform 804 emitted from a 1D transducer array. The axes are the same as in FIG. 8A. The transmit waveform 804 was generated by using the entire aperture (e.g., all of the transducer elements used to transmit ultrasound signals) for low temporal frequency ultrasound signals and reducing the size of the aperture (e.g., reducing the number of transducer elements used to transmit the ultrasound signals) as the temporal frequencies increased.

In the examples in FIGS. 8A and 8B, similar to FIGS. 7A and 7B, a transmit event may include “multiple” transmit beams, each with a different width for each frequency and/or frequency range of the ultrasound signal. In some examples, the maximum width of the transmit beam and/or the narrowest width of the transmit beam may be based, at least in part, on a pitch of the transducer array. For example, transducer arrays with smaller pitch sizes (e.g., small distances between transducer elements or patches) may be capable of adequately sampling high frequencies from wider transmit beams than transducer arrays with larger pitch sizes.

By shaping the transmit beam as disclosed herein, the received signals may include few or no frequencies above the Nyquist limit of the transducer array. Accordingly, in some examples, no filtering of multilines based on steering angle may be necessary to reduce or eliminate grating lobes. In these examples, the multilines may be processed using conventional means to generate an ultrasound image.

As disclosed herein, filtering techniques may be used on received ultrasound signals that filter only multilines and/or steering angles where frequencies are aliased. As disclosed herein, undersampling of the received ultrasound signal may be reduced or avoided by altering a shape of a transmitted ultrasound beam to reduce received spatial frequencies. The techniques disclosed herein may reduce or eliminate grating lobe artifacts caused by aliasing.

In various examples where apparatuses, components, systems and/or methods are implemented using a programmable device, such as a computer-based system or programmable logic, it should be appreciated that the above-described systems and methods can be implemented using any of various known or later developed programming languages, such as “C”, “C++”, “FORTRAN”, “Pascal”, “VHDL” and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software, and/or firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instructions to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and methods may be that conventional medical image systems can be easily upgraded to incorporate the features and advantages of the present systems, devices, and methods.

Of course, it is to be appreciated that any one of the examples, examples or processes described herein may be combined with one or more other examples, examples and/or processes or be separated and/or performed amongst separate devices or device portions in accordance with the present systems, devices and methods.

Finally, the above-discussion is intended to be merely illustrative of the present apparatuses, systems and methods and should not be construed as limiting the appended claims to any particular example or group of examples. Thus, while the present apparatuses, systems, and methods have been described in particular detail with reference to exemplary examples, it should also be appreciated that numerous modifications and alternative examples may be devised by those having ordinary skill in the art without departing from the broader and intended spirit and scope of the present systems and methods as set forth in the claims that follow. Accordingly, the specification and drawings are to be regarded in an illustrative manner and are not intended to limit the scope of the appended claims.

What is claimed is:

1. An ultrasound imaging system comprising:
a transducer array configured for transmitting ultrasound signals, receiving echoes responsive to the ultrasound signals, and providing receive signals corresponding to the echoes for a plurality of multilines; and
a processor configured to:
determine a target steering angle for the transducer array, wherein the target steering angle is based, at least in part, on a pitch of the transducer array and a frequency of the ultrasound signals;
determine a steering angle for individual ones of the plurality of multilines, wherein the steering angle is based, at least in part, on the pitch of the transducer array; and
filter the receive signals corresponding to one or more of the plurality of multilines that have steering angles greater than the target steering angle before processing the receive signals into ultrasound image data.

2. The ultrasound imaging system of claim 1, further comprising a beamformer configured to beamform the receive signals prior to the processor applying the filter.

3. The ultrasound imaging system of claim 1, further comprising a microbeamformer configured to partially beamform the receive signals from groups of transducer elements of the transducer array, wherein the pitch of the transducer array is a distance between the groups of transducer elements.

4. The ultrasound imaging system of claim 1, wherein the target steering angle is based on a Nyquist limit and a value between and including zero and one.

5. The ultrasound imaging system of claim 4, further comprising a user interface, wherein the value is determined by a user input provided via the user interface.

6. The ultrasound imaging system of claim 1, wherein the processor is further configured to compound the receive signals from the individual ones of the plurality of multilines that were filtered with the receive signals from the individual ones of the plurality of multilines that were not filtered.

7. The ultrasound imaging system of claim 1, wherein the processor is further configured to increase a power of the receive signals of the individual ones of the plurality of multilines that were filtered.

8. The ultrasound imaging system of claim 1, further comprising a transmit controller, wherein the transmit controller provides control signals to the transducer array to control an angle of the ultrasound signals, wherein the steering angle for the individual ones of the plurality of multilines is further based, at least in part, on the angle of the ultrasound signals.

9. The ultrasound imaging system of claim 1, wherein the transducer array comprises a two dimensional array.

10. The ultrasound imaging system of claim 1, wherein the transducer array comprises a portion of transducer elements of a plurality of transducer elements that form a larger array.

11. A method, comprising:
transmitting an ultrasound signal with a transducer array;
receiving echoes responsive to the ultrasound signal at the transducer array;
generating receive signals for a plurality of multilines with the transducer array;
determining a target steering angle based, at least in part, on a frequency of the ultrasound signal and a pitch of the transducer array;
determining a steering angle for individual ones of the plurality of multilines; and
filtering the receive signals corresponding to one or more of the plurality of multilines that have steering angles greater than the target steering angle before processing the receive signals into ultrasound image data.

12. The method of claim 11, wherein the steering angles are based, at least in part, on a pitch of the transducer array.

13. The method of claim 11, wherein the steering angles are based, at least in part, on a transmit angle of the ultrasound signal.

14. The method of claim 11, further comprising compounding the receive signals of the plurality of multilines.

15. The method of claim 14, wherein the compounding comprises retrospective transmit beam compounding.

16. The method of claim 11, further comprising beamforming the receive signals prior to filtering.

17. An ultrasound imaging system comprising:
a transducer array configured for transmitting a transmit beam comprising ultrasound signals, receiving echoes responsive to the ultrasound signals, and providing receive signals corresponding to the echoes for a plurality of multilines; and
a controller configured to provide control signals to the transducer array to cause the transducer array to transmit the ultrasound signals such that a width of the transmit beam is adjusted based on frequencies of the ultrasound signals, wherein the width of the transmit beam is wider for low frequencies and narrower for high frequencies.

18. The ultrasound imaging system of claim 17, wherein the width is adjusted by increasing a focal depth of the transmit beam as the frequencies of the ultrasound signals increase.

19. The ultrasound imaging system of claim 17, wherein the width is adjusted by decreasing an aperture of the transducer array as the frequencies of the ultrasound signals increase.

20. The ultrasound imaging system of claim 17, wherein at least one of a maximum width or a minimum width of the transmit beam is based, at least in part, on a pitch of the transducer array.

21. The ultrasound imaging system of claim 17, further comprising a processor configured to process the multilines to generate an ultrasound image.
